(19) United States
(12) Patent Application Publication
(10) Pub. No.: US 2024/0430557 A1
(43) Pub. Date: Dec. 26, 2024

(54) LIGHT SOURCE APPARATUS, ENDOSCOPE SYSTEM, AND LIGHT AMOUNT CONTROL METHOD

(71) Applicant: OLYMPUS MEDICAL SYSTEMS CORP., Tokyo (JP)

(72) Inventors: Kenta AMINO, Tokyo (JP); Nobuyoshi ASAOKA, Ageo-shi (JP); Takayuki SATO, Tokyo (JP)

(73) Assignee: OLYMPUS MEDICAL SYSTEMS CORP., Tokyo (JP)

(21) Appl. No.: 18/825,501

(22) Filed: Sep. 5, 2024

Related U.S. Application Data
(63) Continuation of application No. PCT/JP2022/010578, filed on Mar. 10, 2022.

(51) Int. Cl. H04N 23/56; H04N 23/50; H04N 23/52; H04N 23/55

(52) U.S. Cl. CPC H04N 23/56; H04N 23/52; H04N 23/55; H04N 23/555

(57) ABSTRACT

A light source apparatus includes a first light source, a light sensor, an optical member, a driving circuit configured to move the optical member from a first position to a second position to insert/withdraw the optical member onto/from an optical path of light emitted from the first light source, and a processor. The processor is configured to acquire first information concerning a light amount of light received by the light sensor, acquire second information concerning a position of the optical member while moving from the first position to the second position, and output control information for controlling the first light source on a basis of the first information and the second information.

### CROSS REFERENCE TO RELATED APPLICATION

This application is a continuation application of PCT/JP2022/010578 filed on Mar. 10, 2022, the entire contents of which are incorporated herein by this reference.

### BACKGROUND OF THE INVENTION

### 1. Field of the Invention

The present disclosure relates to a light source apparatus, an endoscope system, and a light amount control method for appropriately controlling light from a plurality of semiconductor light emitting devices.

### 2. Description of the Related Art

Conventionally, endoscope apparatuses including an endoscope for performing observation of a site to be examined and various treatments by inserting an elongated endoscope into a body cavity or the like have been widely used. Such endoscope apparatuses employ a light source apparatus for performing photographing in a body cavity. In recent endoscope apparatuses, a light source apparatus in which semiconductor light emitting devices such as LEDs are employed have been used as a light source in some cases.

Some of such light source apparatuses include a plurality of semiconductor light emitting devices, each of which emits light of different wavelength bands, and radiate multiplexed light obtained by multiplexing light of the plurality of colors as appropriate in accordance with an observation mode such as NBI (registered trademark) (narrow band imaging) or IR (infrared light observation). In order to obtain favorable observation and an endoscope image in an endoscope apparatus, the light source apparatus is controlled so as to keep a color balance (light emission balance) of radiated light constant when radiating multiplexed light of the plurality of colors. The light source apparatus employs in some cases feedback control in which in a case where a light sensor is arranged adjacent to each LED and the light amount of radiated light is changed, the light amount of radiated light from each LED is changed using a result of sensing of the light sensor to attain a predetermined color balance.

For example, Japanese Patent No. 6072369 discloses a technology for, considering that a light sensor fails to accurately sense brightness at a light amount of less than or equal to a predetermined value, raising the light amount once in a case where the light amount is low and then measuring the light amount.

Not only light from the light source but also light reflected off various optical devices, for example, an optical filter, enter the light sensor. In order to obtain a favorable color balance, light amount adjustment considering such reflected light is required. Japanese Patent No. 5393935 discloses a viewpoint of, when sensing the amount of light emitted from a light source, considering not only leaked light but also light reflected off an optical system.

### SUMMARY OF THE INVENTION

A light source apparatus according to an aspect of the present disclosure includes a first light source, a light sensor, an optical member, a driving circuit configured to move the optical member from a first position to a second position to insert/withdraw the optical member onto/from an optical path of light emitted from the first light source, and a processor. The processor is configured to acquire first information concerning a light amount of light received by the light sensor, acquire second information concerning a position of the optical member while moving from the first position to the second position, and output control information for controlling the first light source on a basis of the first information and the second information.

A light source apparatus according to another aspect of the present disclosure includes a light source, a light sensor, an optical member, an optical member driving circuit configured to change the optical member from a first characteristic to a second characteristic to restrict light emitted from the light source, and a processor. The processor is configured to acquire first information concerning a light amount of light received by the light sensor, acquire second information concerning a time period from start of a change of characteristic while the optical member is being changed in characteristic from the first characteristic to the second characteristic, and output control information for controlling the light source on a basis of the first information and the second information.

An endoscope system according to an aspect of the present disclosure includes an endoscope, a first light source, a light sensor, an optical member, a driving circuit configured to move the optical member from a first position to a second position to insert/withdraw the optical member onto/from an optical path of light emitted from the first light source, and a processor. The processor is configured to acquire first information concerning a light amount of light received by the light sensor, acquire second information concerning a position of the optical member while moving from the first position to the second position, and output control information for controlling the first light source on a basis of the first information and the second information.

A light amount control method according to an aspect of the present disclosure is a light amount control method for controlling a light amount of a light source by a processor, including emitting light by the light source, receiving light by a light sensor, acquiring, by the processor, first information concerning a light amount of light received by the light sensor, acquiring, by the processor, second information concerning a position of an optical member to be inserted/removed onto/from an optical path of light emitted from the light source, the position being a position the optical member while moving from a first position to a second position, and outputting, by the processor, control information for controlling the light source on a basis of the first information and the second information.

### Effect of the Invention

The present disclosure has an effect in which a time period required for light amount adjustment can be shortened.

### BRIEF DESCRIPTION OF THE DRAWINGS

FIG. 1 is a configuration diagram showing a light source apparatus according to a first embodiment of the present disclosure;

FIG. 2 is a configuration diagram showing an example of a light source apparatus having a plurality of light sources;

FIG. 3 is a general configuration diagram showing an example of an endoscope and an endoscope apparatus supplied with illumination light from the light source apparatus according to the present embodiment;

FIG. 4 is a general configuration diagram showing an example of an endoscope and an endoscope apparatus supplied with illumination light from the light source apparatus according to the present embodiment;

FIG. 5 is an explanatory diagram showing an example of a configuration of an optical filter 5;

FIG. 6 is an explanatory diagram showing a relationship between a change in positional relationship of the optical filter 5 (a filter portion 5b) with respect to a light flux from a light source 2A when an observation mode is switched and filter reflected light, the horizontal axis indicating the time;

FIG. 7 is a graph showing a change in light amount detected value associated with filter movement, the horizontal axis indicating a filter moved amount, and the vertical axis indicating a light amount detected value based on output of a light sensor 2S;

FIG. 8 is a flowchart showing an example of a case of performing light amount adjustment once when the observation mode is changed;

FIG. 9 is a timing chart showing light amount adjustment when the observation mode is changed;

FIG. 10 is a flowchart showing an example of a case of performing light amount adjustment a plurality of times when the observation mode is changed;

FIG. 11 is a timing chart showing light amount adjustment when the observation mode is changed;

FIG. 12 is a diagram showing a second embodiment of the present disclosure;

FIG. 13 is a diagram showing Modification 1;

FIG. 14 is a diagram showing Modification 1;

FIG. 15 is a diagram showing Modification 2;

FIG. 16 is a configuration diagram showing a third embodiment of the present disclosure; and

FIG. 17 is a diagram describing light amount correction values stored in a memory 6 in the third embodiment.

### DETAILED DESCRIPTION OF THE PREFERRED EMBODIMENTS

Hereinafter, embodiments of the present disclosure will be described in detail with reference to the drawings.

### First Embodiment

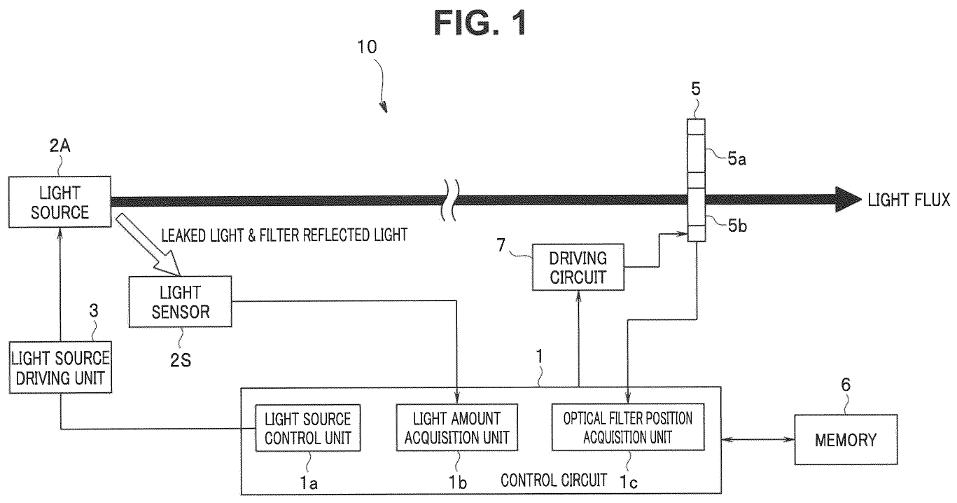

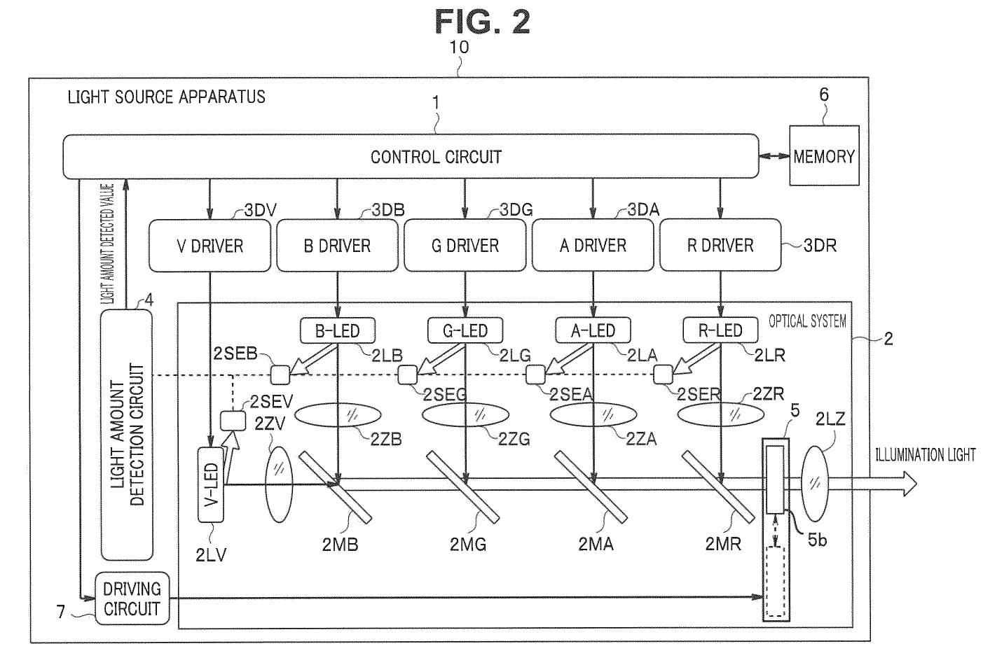

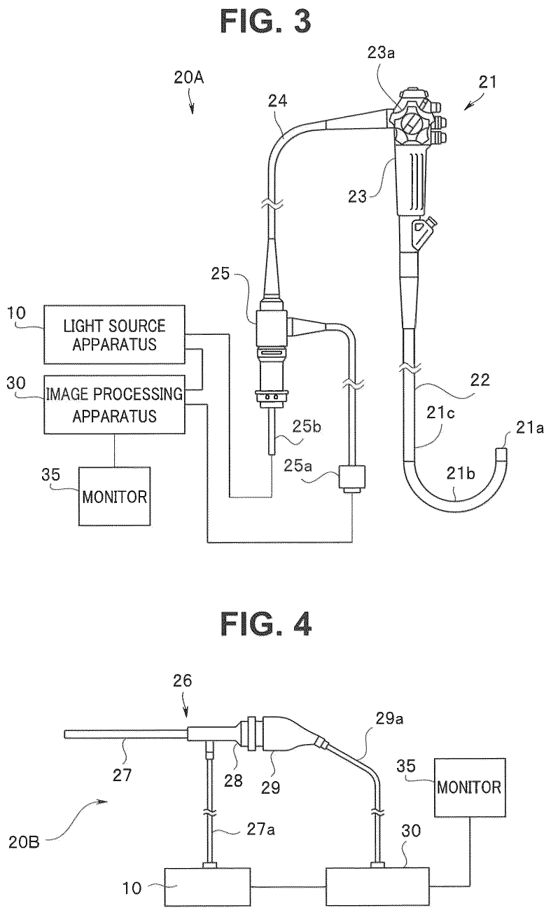

FIG. 1 is a configuration diagram showing a light source apparatus according to a first embodiment of the present disclosure. FIG. 2 is a configuration diagram showing an example of a light source apparatus having a plurality of light sources. FIG. 3 and FIG. 4 are general configuration diagrams showing an example of an endoscope and an endoscope apparatus supplied with illumination light from the light source apparatus according to the present embodiment. Note that in the following description, the drawings based on the embodiments are schematic, and it should be noted that a relationship (dimensional relationship) between the length and width of constituent elements, the ratio between the lengths of respective portions, and the like are different from actual ones. Among the plurality of drawings, portions different in dimensional relationship and ratio are included in some cases. Illustration of some constituent elements is omitted in some cases.

The present embodiment corrects, in accordance with a moved amount, a light amount of a light source measured while an optical filter is moving when an observation mode is switched, thereby enabling the light amount to be accurately found in the process of movement and shortening a time period required for light amount adjustment.

First, an endoscope and an endoscope system supplied with illumination light from the light source apparatus according to the present embodiment will be described with reference to FIG. 3 and FIG. 4.

An endoscope system 20A in FIG. 3 includes a flexible endoscope 21, a light source apparatus 10 that outputs illumination light, an image processing apparatus 30 that performs image pickup processing and the like, and a monitor 35 that displays an endoscope image.

The endoscope 21 includes an insertion section 22, an operation section 23, a universal cord 24, and a scope connector 25. The insertion section 22 to be inserted into a subject is composed of a distal end portion 21a, a bending portion 21b, and an elongated flexible portion 21c. The bending portion 21b composed of a plurality of bending pieces changes an orientation of the distal end portion 21a in accordance with a bending operation of the operation section 23. The flexible portion 21c is formed of a flexible member. The insertion section 22 has a proximal end provided to continue to the operation section 23.

The operation section 23 configures a grasping section to be grasped by an operator, and a bending operation knob 23a that operates the bending portion 21b, and the like are laid out. The universal cord 24 is connected to the operation section 23. The scope connector 25 is provided on the proximal end side of the universal cord 24. The scope connector 25 is provided with an electric connector 25a to be connected to the image processing apparatus 30 and a light receiving rod 25b to be connected to the light source apparatus 10.

Illumination light from the light source apparatus 10 is configured to pass through a light guide inserted through the universal cord 24 and the insertion section 22 from the light receiving rod 25b and is guided to the distal end portion 21a of the insertion section 22. An image pickup apparatus including an image pickup device such as a CMOS sensor not shown is laid out in the distal end portion 21a. An object is irradiated with the illumination light through the distal end portion 21a, and reflected light from the object forms an image on an image pickup plane of the image pickup apparatus. The image pickup apparatus generates an image pickup signal based on an object optical image. This image pickup signal passes through the insertion section 22, the universal cord 24, and the electric connector 25a and is supplied into the image processing apparatus 30. The image processing apparatus 30 subjects the received image pickup signal to predetermined image signal processing to generate a video signal. The image processing apparatus 30 supplies the generated video signal to the monitor 35. An endoscope image is thereby displayed on a display screen of the monitor 35.

An endoscope system 20B in FIG. 4 includes a rigid endoscope 26, the light source apparatus 10, the image processing apparatus 30, and the monitor 35. The rigid endoscope 26 has a rigid insertion section 27, and an eyepiece part 28 is provided on the proximal end side of the insertion section 27. An observation optical system composed of a relay lens not shown for transmitting an object image, and the like and an illumination optical system composed of a light guide not shown and the like are provided in the insertion section 27. A camera 29 is detachably laid out on the eyepiece part 28.

The light source apparatus 10 supplies illumination light to the endoscope 21 via a light guide cable 27a. The object is irradiated with the illumination light, and reflected light from the object forms an image on an image-forming plane of an image pickup device of the camera 29 as an object optical image. The camera 29 supplies an image pickup signal based on the object optical image to the image processing apparatus 30 via an image pickup cable 29a. The image processing apparatus 30 subjects the received image pickup signal to predetermined image signal processing to generate a video signal. The image processing apparatus 30 supplies the generated video signal to the monitor 35 to display an endoscope image on the display screen of the monitor 35.

In FIG. 1, the light source apparatus 10 includes a control circuit 1, the light source 2A, a light source driving unit 3, a light sensor 2S, and a memory 6. The control circuit 1 as a control unit performs overall control over the light source apparatus 10. The control circuit 1 may be configured by a processor including a CPU (central processing unit), a FPGA (field programmable gate array), and the like. The control circuit 1 may operate in accordance with a program stored in a memory not shown to control each unit, or some or all of functions may be implemented by an electronic circuit of hardware.

The light source driving unit 3 is controlled by the light source control unit 1a in the control circuit 1 to control light emission of the light source 2A. The light source 2A is driven by the light source driving unit 3 to emit light. Various light emitting devices such as LEDs (light emitting diodes), LDs (laser diodes), or organic EL (electro luminescence) devices can be employed as the light source 2A. A plurality of types of light emitting devices may be used in a mixed manner as the light source 2A. A light emission amount of the light source 2A is controlled by the light source driving unit 3. Light from the light source 2A is radiated by way of a lens not shown and the like.

In FIG. 1, the optical filter 5 which is an optical member is laid out on a light flux (thick line) of radiated light from the light source 2A. The optical filter 5 includes a filter portion 5a for normal light observation with the endoscope apparatus and a filter portion 5b for special light observation such as NBI (narrow band imaging) and IR (infrared light observation), for example. The optical filter 5 is laid out so as to be driven by the driving circuit 7 to be movable such that the filter portion 5a is interposed or the filter portion 5b is interposed on the light flux. The filter portion 5a may be configured by an open hole that allows direct passage of white light from the light source 2A, for example. The filter portion 5b may have a filter that attenuates an element of predetermined color light. Note that the light flux from the light source 2A has a diameter smaller than the diameter of the filter portions 5a and 5b.

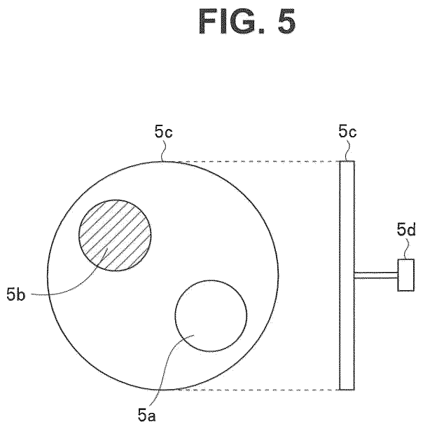

FIG. 5 is an explanatory diagram showing an example of a configuration of the optical filter 5. The left side of FIG. 5 shows a planar shape of the optical filter 5 as seen in a direction parallel to the orientation of the light flux, and the right side of FIG. 5 shows a side surface shape of the optical filter 5 as seen in a direction orthogonal to the orientation of the light flux. The optical filter 5 has a disk-shaped rotation filter frame 5c which is an optical device holding member. The rotation filter frame 5c has, for example, the filter portion 5a formed by the open hole provided in the rotation filter frame 5c and the filter portion 5b formed by a filter that restricts (absorbs) transmission of a predetermined wavelength band for special light observation.

The rotation filter frame 5c has a center attached to a rotation shaft of a stepping motor 5d and is rotatable by the stepping motor 5d within a plane orthogonal to the orientation of the light flux. The driving circuit 7 as an optical member moving unit may be controlled by the control circuit 1 to drive the stepping motor 5d, thereby rotating the rotation filter frame 5c. The filter portion 5a is interposed or the filter portion 5b is interposed on the light flux by means of rotation of the rotation filter frame 5c.

Note that in FIG. 5, the rotation filter frame 5c is configured to rotate 180 degrees so that either the filter portion 5a or the filter portion 5b is switched to be interposed on the light flux. However, by changing the arrangement of the filter portion 5a and the filter portion 5b as appropriate, a rotation angle of the rotation filter frame 5c that switches between the filter portion 5a and the filter portion 5b on the light flux can be set as appropriate.

FIG. 5 shows the example in which the optical filter 5 is configured by the disk shape, and the rotation filter frame 5c is rotated to move the filter portions 5a and 5b. However, the optical filter 5 may be configured to be moved relative to the light flux to interpose the filter portion 5a or 5b on the light flux. FIG. 1 and FIG. 2 show the example in which the optical filter 5 is moved to interpose the filter portion 5a or interpose the filter portion 5b on the light flux. However, the optical path of the light flux may be configured to be changed using a mirror or the like to cause the light flux to pass through the filter portion 5a or 5b.

The light sensor 2S is provided in the vicinity of the light source 2A. The light sensor 2S detects a light amount of entering light from the light sensor 2S and outputs a detected output to a light amount acquisition unit 1b of the control circuit 1. The light amount acquisition unit 1b as a light amount information acquisition unit detects a value of the amount of light produced by the light source 2A on the basis of the detected output of the light sensor 2S. The light source control unit 1a of the control circuit 1 controls the light source driving unit 3 on the basis of a detected value of the light amount obtained by the light amount acquisition unit 1b, thereby performing control such that light of a desired light amount is produced from the light source 2A.

FIG. 2 corresponds to a case where there are a plurality of the light sources 2A. Note that although FIG. 2 shows an example in which LEDs of five colors are employed, LEDs of less than or equal to four colors may be employed, or LEDs of more than or equal to six colors may be employed.

In FIG. 2, the light source apparatus 10 has the control circuit 1 similar to the control circuit 1 in FIG. 1. The light source apparatus 10 has, within the optical system 2, a violet LED (hereinafter referred to as V-LED) 2LV, a blue LED (hereinafter referred to as B-LED) 2LB, a green LED (hereinafter referred to as G-LED) 2LG, an umber LED (hereinafter referred to as A-LED) 2LA, a red LED (hereinafter referred to as R-LED) 2LR (hereinafter representatively referred to as LED 2L in a case where these LEDs do not need to be distinguished) corresponding to the light source 2A in FIG. 1. The V-LED 2LV produces violet light, the B-LED 2LB produces blue light, the G-LED 2LG produces green light, the A-LED 2LA produces umber light, and the R-LED 2LR produces red light.

In the optical system 2, a lens 2ZB and a dichroic filter 2MB are laid out on an optical path of radiated light from LED 2LB, a lens 2ZG and a dichroic filter 2MG are laid out on an optical path of radiated light from LED 2LG, a lens 2ZA and a dichroic filter 2MA are laid out on an optical path of radiated light from LED 2LA, and a lens 2ZR and a dichroic filter 2MR are laid out on an optical path of radiated light from LED 2LR. A lens 2ZV and the dichroic filters 2MB, 2MG, 2MA, and 2MR are laid out on an optical path of radiated light from LED 2LV.

The lenses 2ZV, 2ZB, 2ZG, 2ZA, and 2ZR (hereinafter representatively referred to as a lens 2Z in a case where these lenses do not need to be distinguished) respectively convert radiated light from V-LED 2LV, B-LED 2LB, G-LED 2LG, A-LED 2LA, and R-LED 2LR into substantially parallel light and radiate the substantially parallel light.

The dichroic filter 2MB transmits radiated light from the lens 2ZV and reflects radiated light from the lens 2ZB. The dichroic filter 2MG transmits light from the dichroic filter 2MB and reflects radiated light from the lens 2ZG. The dichroic filter 2MA transmits light from the dichroic filter 2MG and reflects radiated light from the lens 2ZA. The dichroic filter 2MR transmits light from the dichroic filter 2MA and reflects radiated light from the lens 2ZR.

Thus, outputs of the respective LED 2Ls are synthesized (multiplexed) by the dichroic filters 2MB, 2MG, 2MA, and 2MR (hereinafter representatively referred to as a dichroic filter 2M in a case where these dichroic filters do not need to be distinguished). Synthetic light from the dichroic filter 2MR passes through the optical filter 5 and is radiated via the lens 2LZ. Synthetic light from the lens 2LZ is supplied as illumination light to the flexible endoscope 21, the rigid endoscope 26, and the like.

Note that FIG. 2 shows an example in which the driving circuit 7 is controlled by the control circuit 1 to insert/withdraw the optical filter 5 onto/from the optical path of radiated light from the dichroic filter 2MR, so that whether or not the filter portion 5b is interposed on the light flux of radiated light from the dichroic filter 2MR is switched.

The light source apparatus 10 includes a violet driver (hereinafter referred to as V driver) 3DV, a blue driver (hereinafter referred to as B driver) 3DB, a green driver (hereinafter referred to as G driver) 3DG, an umber driver (hereinafter referred to as A driver) 3DA, a red driver (hereinafter referred to as R driver) 3DR (hereinafter representatively referred to as a driver 3D in a case where these drivers do not need to be distinguished) corresponding to the light source driving unit 3 in FIG. 1. The V driver 3DV drives the V-LED 2LV, the B driver 3DB drives the B-LED 2LB, the G driver 3DG drives the G-LED 2LG, the A driver 3DA drives the A-LED 2LA, and the R driver 3DR drives the R-LED 2LR. For example, each of the drivers 3D may control the light emission amount of each LED 2L by current driving of changing a current amount to be supplied to each LED 2L or PWM driving of changing a pulse width of driving pulses.

In the vicinity of each LED 2L configured in the optical system 2, light sensors 2SEV, 2SEB, 2SEG, 2SEA, and 2SER (hereinafter representatively referred to as a light sensor 2SE in a case where these light sensors do not need to be distinguished) each corresponding to the light sensor 2S in FIG. 1 are provided at positions displaced from the optical path of radiated light from each LED 2L. A configuration may be adopted in which a beam splitter not shown is provided between each LED 2L and the corresponding lens 2Z to cause light from each LED 2L to enter the corresponding light sensor 2SE. The light sensors 2SEV, 2SEB, 2SEG, 2SEA, and 2SER mainly detect the light amount of illumination light from V-LED 2LV, B-LED 2LB, G-LED 2LG, A-LED 2LA, and R-LED 2LR, respectively, as indicated by arrows in FIG. 2 and output detected outputs to a light amount detection circuit 4.

The light amount detection circuit 4 corresponds to the light amount acquisition unit 1b in FIG. 1 and detects a light amount value of light produced from each LED 2L on the basis of the detected output of the light sensor 2SE. The light amount detection circuit 4 outputs the light amount detected value of the light produced from each LED 2L to the control circuit 1. The control circuit 1 separately controls the light amount of each LED 2L on the basis of output of the light amount detection circuit 4. Illumination light in a desired light amount and a desired color balance is thereby radiated from the lens 2LZ.

In other words, in the light source apparatus 10 in FIG. 2, light amount adjustment is performed by adjusting a radiated light amount of each LED 2L. By the light amount adjustment, the light amount of synthetic light from the lens 2LZ is adjusted, and color balance adjustment of adjusting a ratio (light amount ratio) of the light emission amount of each LED 2L is performed. The control circuit 1 controls the light amount of each LED 2L while maintaining the ratio (light amount ratio) of the light emission amount of each LED 2L such that an optimum color balance is obtained on the basis of brightness control information from the endoscope system, for example. For example, the control circuit 1 finds light adjustment information corresponding to the light amount value of G-LED 2LG to be set in accordance with the brightness control information from the endoscope system, and for the other V-LED 2LV, B-LED 2LB, A-LED 2LA, and R-LED 2LR, finds light adjustment information in accordance with the light amount value of G-LED 2LG such that a predetermined light amount ratio is reached.

### (Light Reflected by Optical Filter)

In FIG. 1 and FIG. 2, not only leaked light of light produced from the light source 2A or each LED 2L but also reflected light from the optical filter 5 enter the light sensor 2S or each of the light sensors 2SE. In other words, reflected light obtained when light from the light source 2A is reflected off the optical filter 5 enters the light sensor 2S, and reflected light obtained when light from each LED 2L is reflected off the optical filter 5 (hereinafter, the reflected light from the optical filter 5 will be referred to as filter reflected light) enters each of the light sensors 2SE.

Hereinafter, description will be given using the single light source in FIG. 1 as an example for ease of description. The light amount of filter reflected light changes depending on which positions the filter portions 5a and 5b of the optical filter 5 are located relative to the light flux of light from the light source 2A. For example, in a case where a state in which the filter portion 5a is present on the light flux and a state in which the filter portion 5b is present on the light flux are switched by changing the observation mode, the light amount of filter reflected light that enters the light sensor 2S varies. Thus, as described above, conventionally there is a problem in that in order to accurately perform light amount control over the light source 2A, light amount control cannot be carried out until movement of the optical filter 5 associated with the change of observation mode is completed.

Thus, the present embodiment enables the light amount of radiated light from the light source 2A to be accurately detected even during movement of the optical filter 5, thereby shortening a time period required for light amount adjustment to enable a favorable observation image (endoscope image) to be obtained from immediately after the observation mode is switched.

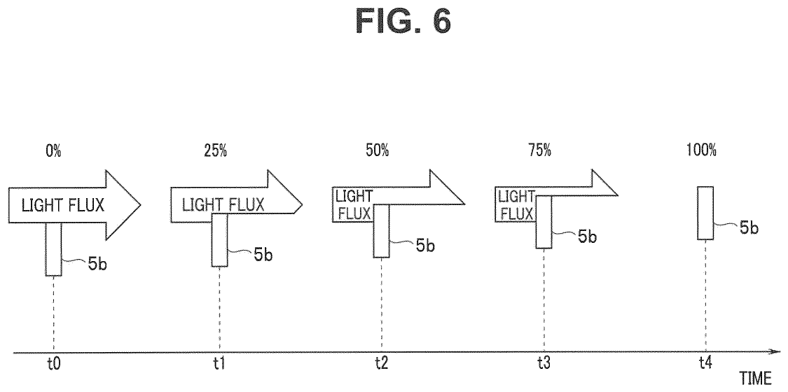

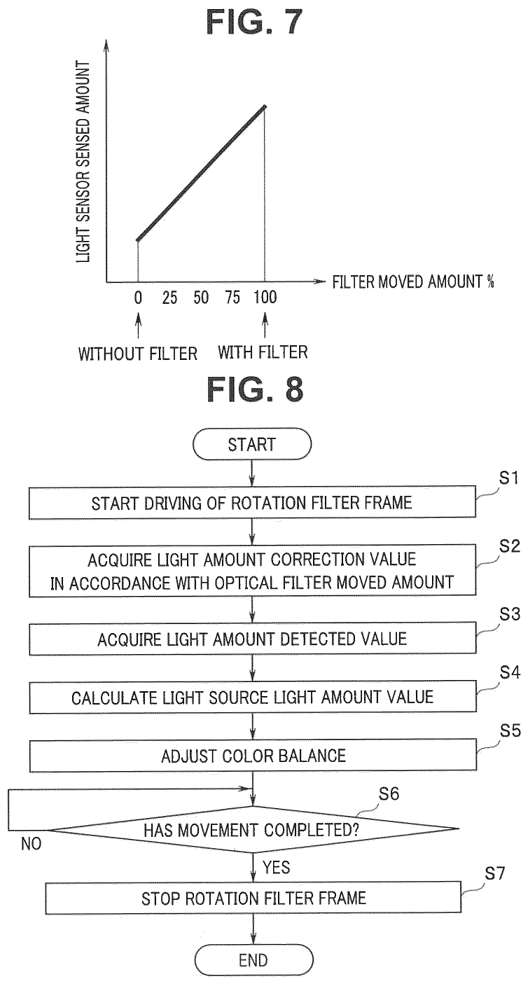

FIG. 6 is an explanatory diagram showing a relationship between a change in positional relationship of the optical filter 5 (the filter portion 5b) relative to the light flux from the light source 2A when the observation mode is switched and filter reflected light, the horizontal axis indicating the time. FIG. 7 is a graph showing a change in light amount detected value associated with filter movement, the horizontal axis indicating the filter moved amount, and the vertical axis indicating the light amount detected value based on output of the light sensor 2S.

Arrows in FIG. 6 indicate a light flux traveling without being blocked by the filter portion 5b. FIG. 6 shows a manner in which the optical filter 5 moves from time points t0 to t4, so that the filter portion 5b is gradually interposed on the light flux. The percentage in FIG. 6 indicates a proportion of an area (hereinafter referred to as a reflection area ratio) in which the light flux is blocked by the filter portion 5b, that is, the degree in which the filter portion 5b is inserted onto the light flux. A state in which the reflection area ratio is 0% at the time point to in FIG. 6 indicates a state in which the filter portion 5b is not interposed on the light flux, and the reflection area ratio of 100% at the time point t4 indicates a state in which the filter portion 5b is interposed on the entire region of the light flux. Similarly, the reflection area ratios of 25%, 50%, and 75% at the time points t1 to t3 respectively indicate the states in which the filter portion 5b is interposed on 25%, 50%, and 75% of the light flux.

The filter portion 5b absorbs light and restricts transmission of light. Light, transmission of which is restricted, is reflected to become filter reflected light. In other words, filter reflected light does not exist at the time point t0. When the light amount of filter reflected light at the time point t4 is assumed as 100%, the light amounts of filter reflected light at the time points t1 to t3 are considered to be approximately 25%, 50%, and 75%.

The reflection area ratio of the filter portion 5b is a value indicating what percentage of the light flux is reflected off the filter portion 5b, and the reflection area ratio is uniquely determined depending on the position of the optical filter 5. Thus, by correcting the light amount detected value based on output of the light sensor 2S using positional information on the optical filter 5 corresponding to the reflection area ratio, that is, the degree of insertion of the filter portion 5b onto the light flux, a current value of the light amount value of radiated light from the light source 2A (hereinafter referred to as a light source light amount value) can be accurately calculated even in the process of movement of the optical filter 5. If the time period required for movement of the optical filter 5 is already known, the light source light amount value after movement of the optical filter 5 is completed can also be found by correcting a current light amount detected value based on output of the light sensor 2S on the basis of the positional information on the optical filter 5.

(Calculation of Light Source Light Amount Value when Optical Filter Moves)

FIG. 7 shows a relationship between such a reflection area ratio (filter moved amount) and the light amount detected value based on output of the light sensor 2S. The characteristics shown in FIG. 7 reveal that the light source light amount value can be calculated even in the process of movement of the optical filter 5.

It is assumed that the filter portion 5b is irradiated with X % of the light flux in the rotary optical filter 5 shown in FIG. 5. In this case, in addition to filter reflected light corresponding to X % of the light flux, an absorptive element of a region of (100-X) % of the light flux is reflected off the rotation filter frame 5c to become filter reflected light. The light source light amount value is found by multiplying the light amount detected value based on output of the light sensor 2S by a predetermined correction value (hereinafter referred to as a reflection correction value) considering such reflection by the filter portion 5b and the rotation filter frame 5c. The present embodiment also enables calculation of an accurate light source light amount value in the process of movement of the optical filter 5 using the reflection area ratio, that is, information on the moved amount of the optical filter 5.

In FIG. 1, the control circuit 1 includes an optical filter position acquisition unit 1c. The optical filter position acquisition unit 1c as a positional information acquisition unit is configured to detect the position of the optical filter 5 using various publicly-known methods to acquire positional information. For example, the optical filter position acquisition unit 1c may detect the position of the optical filter 5 using outputs of various sensors that detect rotation of the rotation filter frame 5c. Alternatively, the optical filter position acquisition unit 1c may detect the position of the optical filter 5 on the basis of a driving pulse number (the number of steps) by the driving circuit 7 that drives the stepping motor 5d. The control circuit 1 calculates the light source light amount value in the process of movement of the optical filter 5 on the basis of the light amount detected value obtained by the light amount acquisition unit 1b and the positional information obtained by the optical filter position acquisition unit 1c.

The light source light amount value during movement of the optical filter 5 can be found by Equation (1) below. Note that positional information in Equation (1) is a value indicating the reflection area ratio.

Light source light amount value=Light amount detected value based on light sensor output×Positional information on the optical filter5×Reflection correction value (1)

The control circuit 1 may calculate the light source light amount value in real time on the basis of Equation (1) above each time the position of the optical filter 5 changes. The control circuit 1 may be configured to cause the memory 6 to store in advance the positional information×the reflection correction value (hereinafter, the value will be referred to as a light amount correction value) in Equation (1) for each position of the optical filter 5. In this case, the control circuit 1 can calculate the light source light amount value by reading out the light amount correction value in accordance with the position of the optical filter 5 from the memory 6 and multiplying the light amount detected value by the light amount correction value as read out.

In the present embodiment, the light source light amount value of each LED 2L is calculated on the basis of Equation (1) above or obtained using information stored in the memory 6. The control circuit 1 controls each of the drivers 3D on the basis of light adjustment information on each LED 2L having been found, thereby obtaining an optimum color balance.

### (Statistical Processing)

In the above description, it has been described that the light amount adjustment may be performed using the light source light amount value in the process of movement of the optical filter 5, or the light source light amount value after movement is completed may be estimated from the light source light amount value in the process of movement of the optical filter 5 to perform the light amount adjustment. The light amount adjustment may be performed by statistical processing of the light source light amount value found in the process of movement.

For example, a plurality of light amount correction values are found between the moved amounts of 0% and 100% of the optical filter 5. Light source light amount values found using these plurality of light amount correction values are processed statistically, and the light sources 2A and LED 2L are controlled. For R-LED 2LR, for example, the light source light amount values when the filter moved amount is 25%, 50%, and 75% shall be found as a, b, and c, respectively. In this case, after movement of the optical filter 5 is completed, an amount of power supply to R-LED 2LR may be found using an arithmetic average (a+b+c)/3, a geometric mean (a²+b²+c²)^{1/2} or a mode value, median, or the like of (a, b, c) as the light source light amount value.

### (Actions)

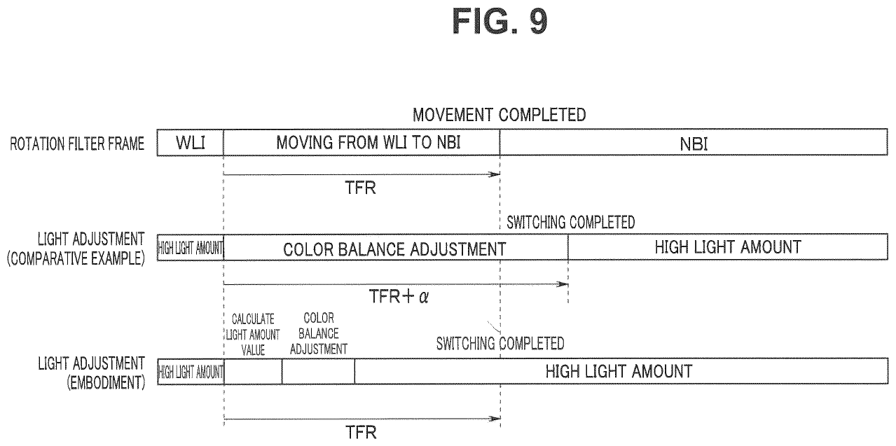

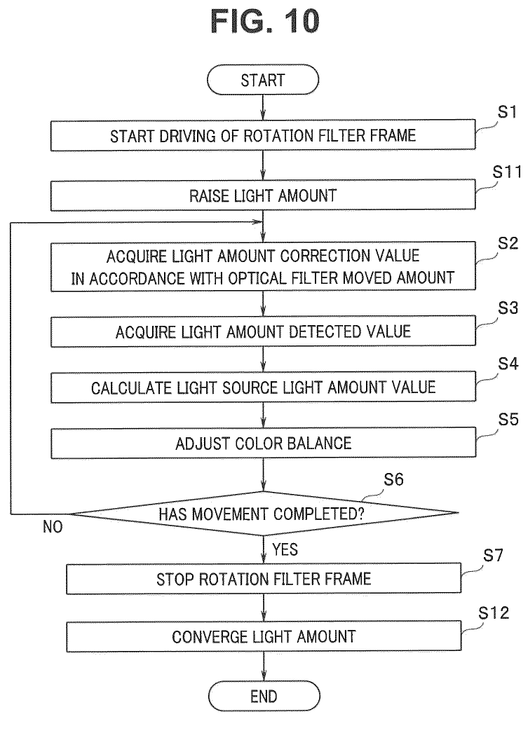

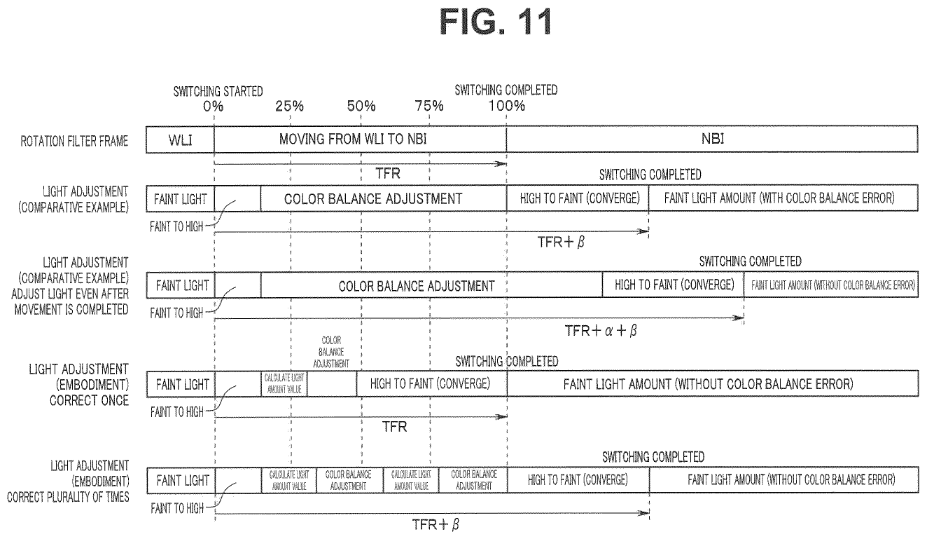

Operation of the embodiment configured in the foregoing manner will now be described with reference to FIG. 8 to FIG. 11. FIG. 8 is a flowchart showing an example of a case of performing light amount adjustment once when the observation mode is changed, and FIG. 10 is a flowchart showing an example of a case of performing light amount adjustment a plurality of times when the observation mode is changed. FIG. 9 and FIG. 11 are timing charts showing light amount adjustment when the observation mode is changed. Note that although the following description of operation will be given using the example of FIG. 2 having a plurality of light sources, operation similar to the operation in the example of FIG. 2 is also performed in the example of FIG. 1 showing the single light source. As described above, color balance adjustment is also performed in light amount adjustment for the plurality of light sources.

### (High Light Amount to High Light Amount)

The observation mode shall now be switched in a state in which the light amount is relatively high (hereinafter referred to as a high light amount). For example, the observation mode shall be switched from the white light observation mode through use of the filter portion 5a to the NBI observation mode through use of the filter portion 5b. FIG. 8 and FIG. 9 show switching in the case of a high light amount. The control circuit 1 starts driving of the rotation filter frame 5c in S1 of FIG. 8. FIG. 9 shows a change in the rotation filter frame 5c on an upper stage, shows light adjustment control in a comparative example on the middle stage, and shows light adjustment control in the first embodiment on a lower stage. As shown in FIG. 9, the proportion in which the filter portion 5b is interposed on the light flux of light from the dichroic filter 2MR changes from the state of 0% to the state of 100% (that is, the optical filter 5 moves) in a transition from the white light observation mode (WLI) to the NBI observation mode (NBI). A time period required for the movement is a time period TFR.

In the comparative example shown on the middle stage of FIG. 9, the light amount value of each LED is found after the movement of the optical filter is completed, and the color balance adjustment is carried out. When a time period required for measurement of the light amount value and color balance adjustment is assumed as a, the time period TFR+α is required for switching of the observation mode in the comparative example.

In contrast, in the present embodiment, calculation of the light amount value is performed from immediately after movement of the optical filter 5 is started for switching of the observation mode. In other words, the optical filter position acquisition unit 1c of the control circuit 1 detects the position of the rotation filter frame 5c and finds positional information. The control circuit 1 reads out a light amount correction value from the memory 6 using the positional information, for example (S2). Each of the light sensors 2SE detects the light amount of each entering light and outputs the detected output to the light amount acquisition unit 1b of the control circuit 1. The light amount acquisition unit 1b finds the light amount detected value on the basis of the detected output of each of the light sensors 2SE (S3). The control circuit 1 multiplies the light amount detected value by the light amount correction value to find the light source light amount value in accordance with a current position of the optical filter 5 for each LED 2L (S4). The control circuit 1 may estimate a light source light amount value after movement of the optical filter 5 is completed from the current light source light amount value.

The light source control unit 1a of the control circuit 1 generates a control signal for rendering the light emission amount of each LED 2L into a defined light emission amount on the basis of the calculated current light source light amount value of each LED 2L or the light source light amount value after movement of the optical filter 5 is completed. This control signal is supplied to each of the drivers 3D, and the driver 3D controls each LED 2L such that light of a desired light amount is produced from each LED 2L (S5).

The control circuit 1 determines whether or not movement of the optical filter 5 has been completed (S6). In a case where movement of the optical filter 5 has not been completed (when determined as NO in S6), the control circuit 1 continues the movement, and in a case where the movement of the optical filter 5 has been completed (when determined as YES in S6), stops rotation of the rotation filter frame 5c (S7).

As shown in FIG. 9, in the present embodiment, calculation of the light source light amount value and color balance adjustment are started immediately after switching of the observation mode is started and are performed for a time period shorter than the time period TFR. Thus, image pickup in an appropriate color balance can be performed simultaneously with completion of the movement of the optical filter 5.

### (Faint Light Amount to Faint Light Amount)

Switching of the observation mode shall now be performed in a state in which a radiated light amount of LED 2L is a faint light amount smaller than a predetermined light amount by which the light sensor 2SE cannot accurately sense brightness. For example, in such a case where a distal end of an endoscope is brought into close contact with an object, the state of a faint light amount is brought about in some cases. FIG. 10 and FIG. 11 show switching in this case. Steps in FIG. 10 identical to the steps in FIG. 8 will be denoted by identical reference characters, and description will be omitted.

Since the light sensor 2SE cannot detect an accurate light amount in the state of a faint light amount, the control circuit 1 exerts control of once raising the light amount into a high light amount (S11) and then measuring the light amount. The observation mode shall now be switched from the white light observation mode through use of the filter portion 5a to the NBI observation mode through use of the filter portion 5b. The control circuit 1 switches the faint light amount to the high light amount in switching of the observation mode, as shown in FIG. 11. In this state, the control circuit 1 starts driving of the rotation filter frame 5c in S1 of FIG. 9.

FIG. 11 shows a change in the rotation filter frame 5c on the uppermost stage, shows light adjustment control in a comparative example on the second and third stages, and shows light adjustment control in the embodiment on the fourth and fifth stages. As shown in FIG. 11, a time period required for the filter portion 5b to move onto the light flux of light from the dichroic filter 2MR to change from the state in which the reflection area ratio is 0% to the state in which the reflection area ratio is 100% in order to transition from the white light observation mode (WLI) to the NBI observation mode (NBI) is the time period TFR.

The comparative example shown on the second stage of FIG. 11 finds the light amount value of each LED in the process of movement of the optical filter and carries out the color balance adjustment. After the movement of the optical filter is completed, converging control of returning the high light amount to the faint light amount is performed. Assuming that a time period required for returning (converging) the high light amount to the faint light amount is β, the time period required for switching of the observation mode is the time period TFR+B. Since the light amount value of each LED is found in the process of movement of the optical filter, the found light amount value is inaccurate due to an influence of filter reflected light, as a result of which the color balance has an error.

The comparative example shown on the second stage of FIG. 11 carries out the color balance adjustment even after the movement of the optical filter is completed. Control of returning the high light amount to the faint light amount is performed after the color balance adjustment. In this case, even though there is no error in color balance, the time period required for switching of the observation mode becomes the time period TFR+α+B.

In contrast, in the present embodiment, calculation of the light source light amount value is performed from immediately after movement of the optical filter 5 is started for switching of the observation mode (S2 to S4). The current light source light amount value or the light source light amount value after the movement of the optical filter 5 is completed is thus found. The example on the fourth stage of FIG. 11 corresponds to the flow of FIG. 8, and the color balance adjustment is performed only once. After the color balance adjustment finishes, the control of returning the high light amount to the faint light amount is performed (S12). As a result, as shown on the fourth stage of FIG. 11, the time period required for switching of the observation mode can be shortened to the time period TFR without causing an error in color balance.

The example on the fifth stage of FIG. 11 corresponds to the flow of FIG. 9. In this case, when it is determined in S6 that the movement of the optical filter 5 has not been completed (when determined as NO in S6), processing from S2 to S5 is repeated. In other words, calculation of the light source light amount value and the color balance adjustment are repeated, and more accurate color balance adjustment is carried out. Note that in this case, the time period required for switching of the observation mode becomes the time period TFR+B.

As described, the present embodiment enables the light amount of radiated light from the light source 2A to be accurately detected even during the movement of the optical filter 5, thereby shortening the time period required for light amount adjustment to enable a favorable observation image (endoscope image) to be obtained from immediately after the observation mode is switched.

Note that in the above description, it has been described that the light amount control is carried out using the light source light amount value found during the movement of the optical filter 5 and observation is performed after a transition of the observation mode. It may be configured such that the light source 2A is caused to emit light in a normal light intensity which is not faint light in the process of the observation, the light source light amount value is re-measured using the light amount correction value corresponding to the moved amount of 100%, and the light amount control is then carried to continue observation using the re-measured light source light amount value.

### Second Embodiment

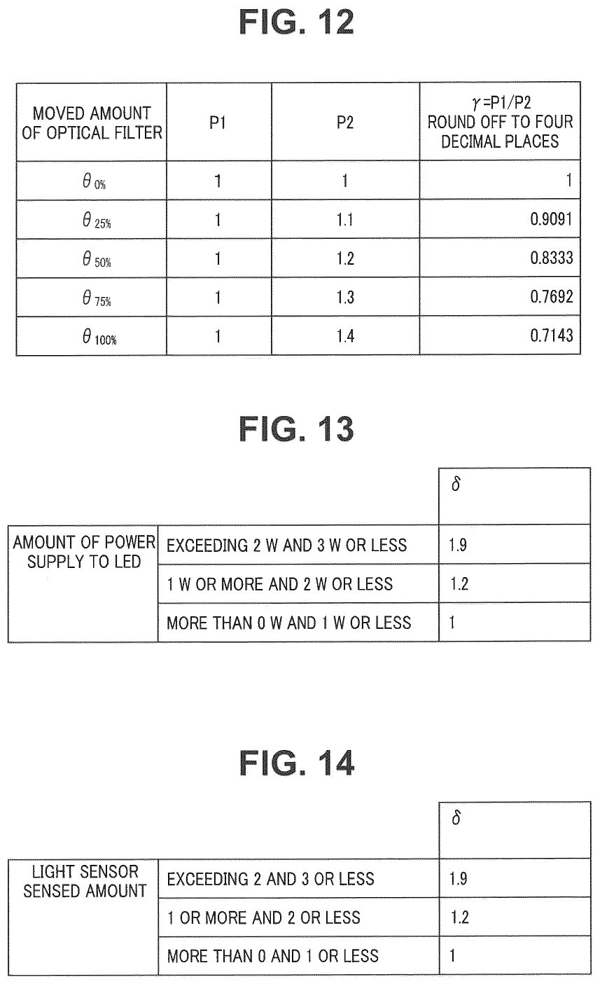

FIG. 12 is a diagram showing a second embodiment of the present disclosure. The second embodiment indicates an example of a specific manner of finding the light amount correction value. The present embodiment has a hardware configuration similar to the hardware configuration of the first embodiment. The present embodiment is also similar to the first embodiment in terms of the calculation formulas of the current light source light amount value, estimation of the light source light amount value after switching of the observation mode, the statistical processing of the light source light amount value, and methods for the light amount adjustment and the color balance adjustment.

FIG. 12 describes light amount correction values stored in the memory 6. In the diagram, P1 indicates a light amount value obtained from a measurement result of each of the light sensors 2SE in the case where the filter portion 5b is not interposed on the light flux of radiated light from the dichroic filter 2MR, and P2 indicates a light amount value obtained from the measurement result of each of the light sensors 2SE in the case where the filter portion 5b is interposed on the light flux of radiated light from the dichroic filter 2MR. The example of FIG. 12 indicates P2 in a case where P1 is assumed as 1 for each moved amount.

The moved amount (%) in FIG. 12 is a value indicating what percentage of the light flux in which the filter portion 5b is interposed. In the case where the moved amount is 0%, P1=P2=1 holds, and a light amount correction value γ is 1. As the moved amount increases, the filter reflected light increases, and P2 becomes larger. The light amount correction value γ becomes a smaller value as the moved amount becomes larger.

The light amount correction value γ is found by a ratio between the light amount value P1 when the filter is not interposed and the light amount value P2 when the filter is interposed and is expressed as γ=P1/P2. The control circuit 1 finds the light amount value P1 obtained from the measurement result of each of the light sensors 2SE in the state in which the filter portion 5b is not interposed. The control circuit 1 moves the optical filter 5 in a state in which power supplied to each LED 2L is constant without changing control over each of the light sensors 2SE and finds the light amount value P2 obtained from the measurement result of each of the light sensors 2SE while changing the moved amount. The control circuit 1 calculates the light amount correction value γ by computation of P1/P2. The control circuit 1 causes the memory 6 to store the moved amount and the light amount correction value γ in association with each other as shown in FIG. 12.

When the observation mode is switched, the control circuit 1 finds the moved amount of the optical filter 5. The control circuit 1 reads out the light amount correction value from the memory 6 using the found moved amount. The control circuit 1 finds the light amount detected value on the basis of the detected output of each of the light sensors 2SE and multiplies the light amount detected value by the light amount correction value to find the light source light amount value in accordance with the current position of the optical filter 5 for each LED 2L. The control circuit 1 may estimate the light source light amount value after the movement of the optical filter 5 is completed from the current light source light amount value. The control circuit 1 may find the light source light amount value after the movement of the optical filter 5 is completed by the aforementioned statistical method.

The control circuit 1 controls the light amount of each LED 2L on the basis of the found light source light amount value. This enables image pickup in an accurate color balance to be performed from immediately after the movement of the optical filter 5 is completed.

Other components and actions are similar to the components and actions of the first embodiment.

### Modification 1

FIG. 13 and FIG. 14 are diagrams showing Modification 1. In FIG. 12, the light amount correction value γ is calculated on the basis of the light amount values P1 and P2. However, it can also be considered that the light amount correction value γ depends on emission intensities of LEDs. FIG. 13 considers this case, and the light amount correction value γ is found by γ=(P1/P2)×δ. The amount of power supply to each LED 2L and the value of a correction value δ shown in FIG. 13 are stored in the memory 6 in association with each other.

In the example of FIG. 13, δ=1.9 holds in a case where the amount of power supply to LED 2L is more than 2 W and 3 W or less, δ=1.2 holds in a case where the amount of power supply to LED 2L is 1 W or more and 2 W or less, and δ=1 holds in a case where the amount of power supply to LED 2L is more than 0 W and 1 W or less. In other words, the light source light amount value is found as a larger value as the amount of power supply to LED 2L is larger.

FIG. 14 considers that the light amount correction value γ depends on a sensed amount of the light sensor 2SE. In this case, the light amount correction value γ is also found by γ=(P1/P2)×δ. The sensed amount of each of the light sensors 2SE and the value of the correction value δ shown in FIG. 14 are stored in the memory 6 in association with each other.

In the example of FIG. 14, δ=1.9 holds in a case where the sensed amount of the light sensor 2SE is 2 or more and 3 or less, δ=1.2 holds in a case where the sensed amount of the light sensor 2SE is 1 or more and 2 or less, and δ=1 holds in a case where the sensed amount of the light sensor 2SE is more than 0 and 1 or less. In other words, the light source light amount value is found as a larger value as the sensed amount of the light sensor 2SE is larger.

Other components and actions are similar to the components and actions of the second embodiment.

### Modification 2

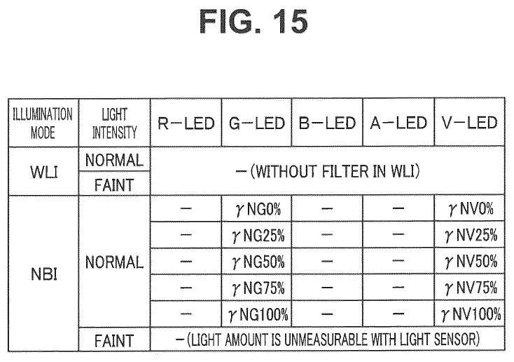

FIG. 15 is a diagram showing Modification 2. Although not being mentioned in the description with reference to FIG. 12 to FIG. 14, it is considered that the filter reflected light mainly includes a particular wavelength element in accordance with a characteristic of the filter portion 5b. For example, the filter portion 5b employed in the NBI observation mode has a characteristic of absorbing green light and violet light in some cases, and it is considered that filter reflected light of these color lights is obtained.

Therefore, these color lights should only be considered for the light amount correction value γ. FIG. 15 shows light amount correction values in this case. In the example of FIG. 15, light amount correction values for G-LED 2LG with the moved amounts of 0%, 25%, 50%, 75%, and 100% are expressed by γNG0%, γNG25%, γNG50%, γNG75%, and γNG100%, respectively. Light amount correction values for V-LED 2LV with the moved amounts of 0%, 25%, 50%, 75%, and 100% are expressed by γNG0%, γNG25%, γNG50%, γNG75%, and γNG100%, respectively.

Note that it is not necessary to consider the influence of filter reflected light for R-LED 2LR, B-LED 2LB, and A-LED 2LA. The filter portion 5a to be used in the white light observation (WLI) mode does not have the characteristic of absorbing light (without a filter). Even in the narrow band observation (NBI) mode, the light amount is unmeasurable for faint light, and light amount correction values are not set. Information shown in FIG. 15 is stored in the memory 6.

The control circuit 1 multiplies the light amount detected value based on the detected output of the light sensor 2SE by the light amount correction values γNG0% to γNG100% in accordance with the moved amounts, thereby finding the light source light amount value of G-LED 2LG. The control circuit 1 also multiplies the light amount detected value based on the detected output of the light sensor 2SEV by the light amount correction values γNV0% to γNV100% in accordance with the moved amounts, thereby finding the light source light amount value of V-LED 2LV. For R-LED 2LR, B-LED 2LB and A-LED 2LA, light amount values based on the detected outputs of the light sensor 2SER, the light sensor 2SEB, and the light sensor 2SEA are used as light source light amount values.

The method for controlling each LED 2L using the found light source light amount values is similar to the method of the above-described second embodiment.

Other components and actions are also similar to the components and actions of the second embodiment.

### Third Embodiment

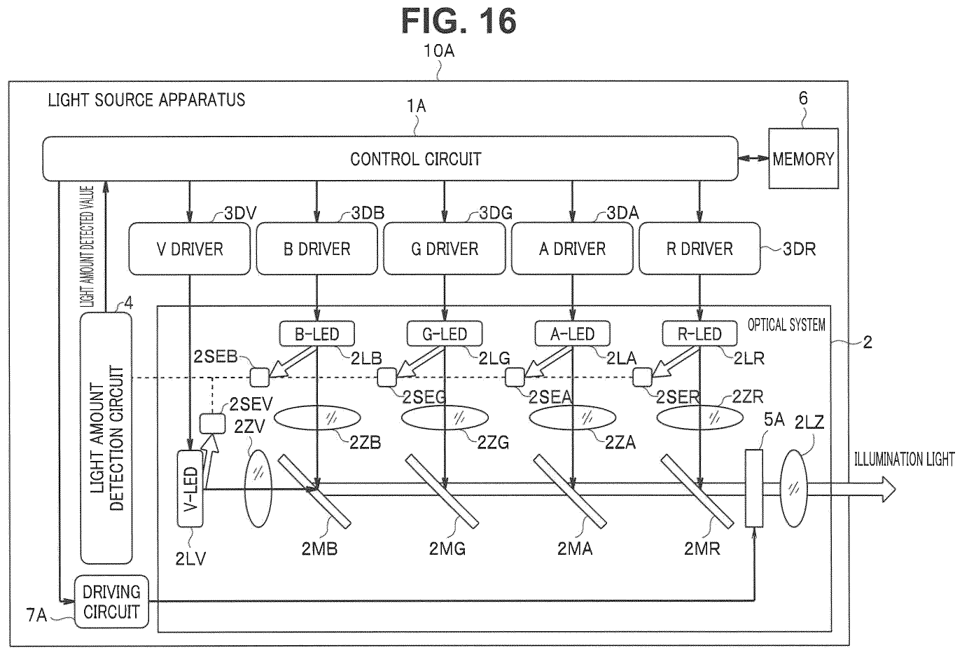

FIG. 16 is a configuration diagram showing a third embodiment of the present disclosure. In FIG. 16, constituent elements identical to the constituent elements in FIG. 1 will be denoted by identical reference characters, and description will be omitted. A light source apparatus 10A in the present embodiment is different from the light source apparatus 10 of the first and second embodiments in that an electro-optic filter 5A is employed instead of the optical filter 5.

In the above-described first and second embodiments, the example of physically moving the optical filter 5 to switch the observation mode has been described. However, the present embodiment enables the observation mode to be switched by changing the characteristic of the electro-optic filter 5A which is an optical device. The electro-optic filter 5A is electrically controlled by a driving circuit 7A to be changed in its optical characteristic. For example, a filter that can increase/decrease the rate of absorption of a wavelength in accordance with an applied voltage because of the Franz-Keldysh effect may be employed as the electro-optic filter 5A.

A control circuit 1A performs overall control over the light source apparatus 10A. The control circuit 1A may be configured by a processor including a CPU, a FPGA, and the like. The control circuit 1A may operate in accordance with a program stored in the memory not shown to control each unit, or some or all of functions may be implemented by an electronic circuit of hardware. The control circuit 1A has functions similar to the functions of the control circuit 1 in the first and second embodiments. The control circuit 1A has a time period information acquisition unit that acquires an elapsed time period in the change of characteristic of the electro-optic filter 5A instead of the optical filter position acquisition unit 1c in FIG. 1.

The control circuit 1A is capable of controlling the driving circuit 7A to switch the characteristic of the electro-optic filter 5A between a characteristic suitable for the white light observation and a characteristic suitable for the special light observation. The electro-optic filter 5A also requires a predetermined time period for the change of the characteristic caused by a change in applied voltage. Therefore, when a predetermined time period elapses from a time point at which the change in applied voltage from the driving circuit 7A for switching the observation mode is started, switching of the observation mode is completed. The characteristic of the electro-optic filter 5A gradually changes between the start of switching of the observation mode and completion of switching, and the light amount of filter reflected light also changes in the period. Therefore, even in the case where the electro-optic filter 5A is employed, illumination light in an appropriate light amount and color balance can be radiated in a short time period after switching of the observation mode is completed by acquiring the light source light amount value in the process of changing the characteristic of the electro-optic filter 5A.

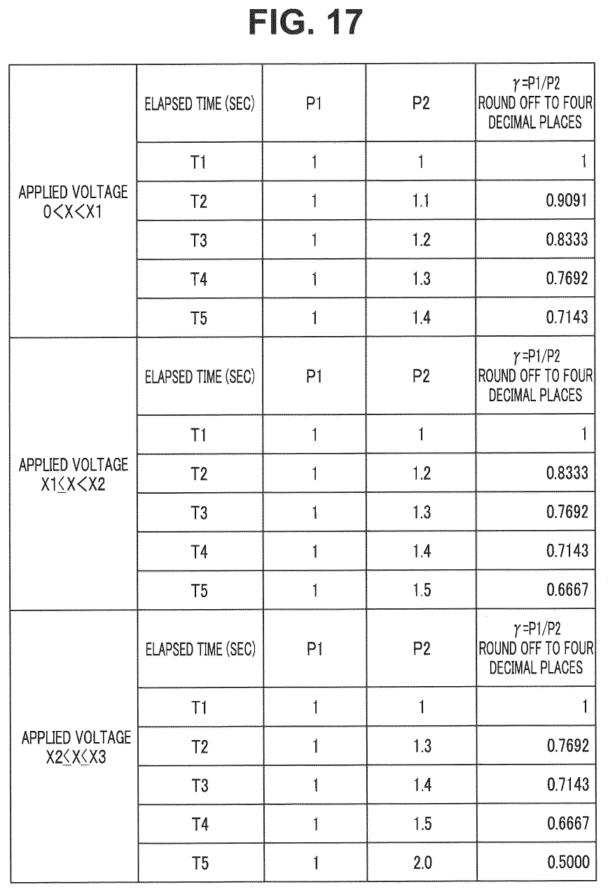

FIG. 17 is a diagram describing light amount correction values stored in the memory 6 in the third embodiment. In the diagram, P1 indicates a light amount value obtained from the measurement result of each of the light sensors 2SE in the case where the electro-optic filter 5A has the characteristic for the white light observation of directly radiating entering light, and P2 indicates light amount values obtained from the measurement result of each of the light sensors 2SE at each time point until the electro-optic filter 5A changes to a characteristic required for the special light observation in a control state identical to the control state of each LED 2L when P1 is measured. The example of FIG. 16 shows P2 in a case where P1 is assumed as 1 every elapsed time period from the start of change.

The elapsed time period (sec) in FIG. 17 shows an elapsed time period in which the electro-optic filter 5A changes from the characteristic for the white light observation to the characteristic for the special light observation. T1 is an elapsed time period of 0 second, and T5 is a time period until a transition is completely made to the characteristic for the special light observation. In the elapsed time period T1, P1=P2=1 holds, and the light amount correction value γ is 1. As the elapsed time period increases, the filter reflected light increases, and P2 becomes larger. The light amount correction value γ becomes a smaller value as the elapsed time period becomes longer.

The light amount correction value γ is found by a ratio between the light amount value P1 in the case where the electro-optic filter 5A has the characteristic for the white light observation and the light amount value P2 in the case where the electro-optic filter 5A changes to the characteristic for the special light observation and is expressed as γ=P1/P2. The control circuit 1A finds the light amount value P1 obtained from the measurement result of each of the light sensors 2SE in the state in which the electro-optic filter 5A is set at the characteristic for the white light observation. The control circuit 1A changes the applied voltage to the electro-optic filter 5A without changing control over each of the light sensors 2SE and finds the light amount value P2 obtained from the measurement result of each of the light sensors 2SE every elapsed time period after the change in applied voltage is started. The control circuit 1A calculates the light amount correction value γ by computation of P1/P2.

It is also considered that the light amount correction value γ changes in accordance with a range of the applied voltage to the electro-optic filter 5A. Considering this case, the light amount correction value γ is found for each range of the applied voltage. The control circuit 1A causes the memory 6 to store the elapsed time period and the light amount correction value γ in association with each other for each range of the applied voltage as shown in FIG. 17.

When the observation mode is switched, the control circuit 1A finds an elapsed time period from the start of the change in applied voltage to the electro-optic filter 5A. The control circuit 1A reads out the light amount correction value from the memory 6 using the found elapsed time period. The control circuit 1A finds the light amount detected value on the basis of the detected output of each of the light sensors 2SE and multiplies the light amount detected value by the light amount correction value to find for each LED 2L the light source light amount value in accordance with the current characteristic of the electro-optic filter 5A. The control circuit 1A may estimate the light source light amount value after the change of characteristic of the electro-optic filter 5A is completed from the current light source light amount value. The control circuit 1A may find the light source light amount value after the change of characteristic of the electro-optic filter 5A is completed by the aforementioned statistical method.

The control circuit 1A controls the light amount of each LED 2L on the basis of the found light source light amount value. This enables image pickup in an accurate color balance to be performed from immediately after the change of characteristic of the electro-optic filter 5A is completed.

Other components and actions are similar to the components and actions of the first and second embodiments.

The present disclosure is not limited to each of the above-described embodiments as they are, and constituent elements can be modified and embodied within the range not departing from the spirit of the disclosure in a practical phase. Appropriate combination of a plurality of constituent elements disclosed in each of the above-described embodiments enables various inventions to be formed. For example, some constituent elements of all the constituent elements described in the embodiments may be eliminated. Constituent elements according to different embodiments may be combined as appropriate.

What is claimed is:

1. A light source apparatus comprising:
a first light source;
a light sensor;
an optical member;
a driving circuit configured to move the optical member from a first position to a second position to insert/withdraw the optical member onto/from an optical path of light emitted from the first light source; and
a processor, wherein
the processor is configured to
acquire first information concerning a light amount of light received by the light sensor,
acquire second information concerning a position of the optical member while moving from the first position to the second position, and
output control information for controlling the first light source on a basis of the first information and the second information.

2. The light source apparatus according to claim 1, wherein the processor is configured to find a first light amount of light radiated from the first light source on a basis of the first information and third information obtained by correcting, on a basis of the second information, a correction value for removing an influence to be caused by reflected light of the optical member in the light amount of the light received by the light sensor and output control information for controlling the first light source on a basis of the first light amount as found.

3. The light source apparatus according to claim 1, wherein the second information indicates a degree of insertion of the optical member onto the optical path.

4. The light source apparatus according to claim 2, further comprising a memory configured to store information indicating a correspondence relationship between the second information and the third information.

5. The light source apparatus according to claim 4, wherein the memory is configured to store the third information found on a basis of a comparison between the first information in a case where the optical member is located at the first position and the first information in a case where the optical member is located from the first position to the second position in a state in which power to the first light source is constant.

6. The light source apparatus according to claim 1, further comprising a second light source.

7. The light source apparatus according to claim 6, further comprising a memory configured to store information indicating a correspondence relationship between the second information and the third information obtained by correcting, on a basis of the second information, a correction value for removing an influence to be caused by reflected light of the optical member in the light amount of the light received by the light sensor, separately for the first light source and the second light source.

8. The light source apparatus according to claim 1, wherein the optical member is supported by a rotation frame member.

9. The light source apparatus according to claim 8, wherein the processor is configured to acquire the second information on a basis of a rotation angle of the rotation frame member.

10. The light source apparatus according to claim 1, wherein the optical member is an optical filter.

11. The light source apparatus according to claim 7, wherein the processor is configured to refer to the memory using the second information as acquired to independently control the first light source and the second light source.

12. The light source apparatus according to claim 11, wherein the processor is configured to complete control over the first light source and the second light source until the optical member reaches the second position.

13. The light source apparatus according to claim 11, wherein the processor is configured to control the first and second light sources on a basis of a result obtained by statistical processing for the third information acquired while the optical member is moving from the first position to the second position.

14. The light source apparatus according to claim 13, wherein the processor is configured to employ an arithmetic average, a geometric mean, a mode value, or a median as the statistical processing.

15. The light source apparatus according to claim 13, wherein the processor is configured to newly re-acquire the third information in a state in which the optical member is located at the second position during control over the first and second light sources based on the third information acquired while the optical member is moving from the first position to the second position and then control the first and second light sources on a basis of the third information as re-acquired.

16. A light source apparatus comprising:
a light source;
a light sensor;
an optical member;
an optical member driving circuit configured to change the optical member from a first characteristic to a second characteristic to restrict light emitted from the light source; and
a processor, wherein
the processor is configured to
acquire first information concerning a light amount of light received by the light sensor,
acquire second information concerning a time period from start of a change of characteristic while the optical member is being changed in characteristic from the first characteristic to the second characteristic, and
output control information for controlling the light source on a basis of the first information and the second information.

17. An endoscope system comprising:
an endoscope;
a first light source;
a light sensor;
an optical member;
a driving circuit configured to move the optical member from a first position to a second position to insert/withdraw the optical member onto/from an optical path of light emitted from the first light source; and
a processor, wherein
the processor is configured to
acquire first information concerning a light amount of light received by the light sensor,
acquire second information concerning a position of the optical member while moving from the first position to the second position, and
output control information for controlling the first light source on a basis of the first information and the second information.

18. A light amount control method for controlling a light amount of a light source by a processor, comprising:
emitting light by the light source;
receiving light by a light sensor;
acquiring, by the processor, first information concerning a light amount of light received by the light sensor;
acquiring, by the processor, second information concerning a position of an optical member to be inserted/removed onto/from an optical path of light emitted from the light source, which is the position of the optical member while moving from a first position to a second position; and
outputting, by the processor, control information for controlling the light source on a basis of the first information and the second information.
